(19) United States
(12) Patent Application Publication
(10) Pub. No.: US 2005/0227063 A1
(43) Pub. Date: Oct. 13, 2005

(54) Plasmon nanoparticles and pixels, displays and inks using them

(75) Inventor: Nabil M. Lawandy, Saunderstown, RI (US)

Correspondence Address: LOWRIE, LANDO & ANASTASI, RIVERFRONT OFFICE, ONE MAIN STREET, ELEVENTH FLOOR, CAMBRIDGE, MA, 02142, US

(73) Assignee: Solaris Nanosciences, Inc., Providence, RI (US)

(21) Appl. No.: 11/091,702

(22) Filed: Mar. 28, 2005

Related U.S. Application Data
(60) Provisional application No. 60/556,765, filed on Mar. 26, 2004.

(51) Int. Cl. C12P 21/06

(52) U.S. Cl. 428/323

(57) ABSTRACT

A pixel that includes a plurality of plasmon nanoparticles is provided. In certain examples, the pixel is configured to transmit or to reflect a variable wavelength of light with varying concentrations of plasmon nanoparticles. Displays including the pixels are also disclosed. An ink which includes plasmon nanoparticles is also provided.

### PRIORITY APPLICATION

This application claims the priority benefit under 35 U.S.C. § 119(e) to U.S. Provisional Application No. 60/556,765 filed on Mar. 26, 2004 and entitled “FIELD-AGGREGATION DISPLAY,” the entire disclosure of which is hereby incorporated by reference herein for all purposes.

### FIELD OF THE TECHNOLOGY

Certain examples of the technology disclosed herein relate to pixels, displays and inks. More particularly, certain examples disclosed herein relate to the use of plasmon nanoparticles to tune the color of a pixel, a display or an ink.

### BACKGROUND

Localized surface plasmon have been observed since the Romans who used gold and silver nanoparticles to create colored glass objects such as the Lycurgus Cup (4^{th} Century AD). A gold sol in the British museum, created by Michael Faraday in 1857, is still exhibiting its red color due to the plasmon resonance at ˜530 nm. In more recent times, localized plasmons have been observed on rough surfaces and in engineered nanostructures and have led to the observation and exploitation of Surface Enhanced Raman Scattering (SERS) and new tuneable plasmon structures with potential applications in biology and medicine.

### SUMMARY

In accordance with a first aspect, a pixel is disclosed. In certain examples, the pixel comprises a plurality of plasmon nanoparticles. In other examples, the pixel may be configured to transmit or to reflect a variable wavelength of light with varying concentrations of plasmon nanoparticles. In some examples, the color response of the plasmon nanoparticles may be tuned within the pixel. In some examples, the wavelength of the light may vary over the entire visible wavelength range (e.g., about 380 nm to about 800 nm) or other selected wavelength range. In certain examples, the plasmon nanoparticles may be encapsulated to form at least one plasmon nanoparticle filled microcapsule.

In accordance with another aspect, a display comprising one or more pixels comprising a plurality of plasmon nanoparticles is provided. In certain examples, each of the pixels comprises a plurality of nanoparticles. In some examples, at least one pixel of the display may be configured to transmit or to reflect a variable wavelength of light with varying concentrations of plasmon nanoparticles. In certain examples, the plasmon nanoparticles may be encapsulated to form plasmon nanoparticles filled microcapsules. In some examples, each of the microcapsules within a pixel may be individually tuned, e.g., the color of each pixel may be individually controlled, to provide a desired color response.

In accordance with another aspect, an ink comprising a plurality of plasmon nanoparticles is provided. In certain examples, the color of the ink is continuously variable over a wavelength range with varying plasmon nanoparticles or with varying plasmon nanoparticle concentrations. In some examples, the ink transmits or reflects light in a visible wavelength range, an infrared wavelength range or in an ultraviolet wavelength range.

In accordance with an additional aspect, a method of controlling color absorption or color transmission of a pixel is provided. In certain examples, the method includes perturbing plasmon nanoparticles in a pixel to control color absorption or color transmission of the pixel along a wavelength range. In certain examples, the plasmon nanoparticles are perturbed by application of an electric field, a magnetic field, or other suitable stimulus.

In accordance with another aspect, a method of facilitating control of a pixel in a display is provided. In certain examples, the method includes providing a pixel configured to transmit or to reflect a variable wavelength of light with varying concentrations of plasmon nanoparticles.

These and other aspects and features are further described in more detail below, and additional aspects and features that use the technology described herein will be readily selected by the person of ordinary skill in the art, given the benefit of this disclosure.

### BRIEF DESCRIPTION OF THE DRAWINGS

Certain examples are described below with reference to the accompanying figures in which:

FIG. 1 is a schematic of a core-shell plasmon nanoparticle, in accordance with certain examples;

FIG. 2 is a schematic of a single metal plasmon nanoparticle, in accordance with certain examples;

FIG. 3 is a schematic of pixel containing multiple plasmon nanoparticles of FIG. 1. and without application of a perturbation, in accordance with certain examples;

FIG. 4 is a schematic of the pixel of FIG. 3 but shown with application of a perturbation, in accordance with certain examples;

FIG. 5 is a plurality of pixels of FIG. 2 with varying levels of plasmon nanoparticle concentrations to exhibit a pattern of colored pixels, in accordance with certain examples;

FIG. 6 is a pixel containing multiple encapsulated groupings of plasmon nanoparticles, in accordance with certain examples; and

FIGS. 7A and 7B are schematics of a concentrating agent with attached plasmon nanoparticles in both an uncoiled state (FIG. 7A) and coiled state (FIG. 7B), in accordance with certain examples.

It will be recognized by the person of ordinary skill in the art, given the benefit of this disclosure, that the size or dimensions of certain features or components on the figures may have been enlarged or distorted relative to other features or components to provide a more user friendly description of the illustrative embodiments disclosed herein.

### DETAILED DESCRIPTION

Certain examples disclosed herein provide significant advantages over existing pixels, displays and inks including, for example, the ability to tune or adjust the color of an individual pixel over a wide wavelength range, e.g., continuously over the entire visible wavelength range, the ability to tune individual components in a pixel and the possibility of assembling displays that are more color responsive, cheaper to produce, provide better contrast and viewing angles and the like. These and other advantages of the illustrative pixels, displays and inks described herein will be readily recognized by the person of ordinary skill in the art, given the benefit of this disclosure.

In accordance with certain examples, certain particles are known to exhibit plasmon resonances which are a function of shape, structure, and the optical properties of the materials and surrounding material responses. Such particles are referred to in some instances herein as “plasmon supporting nanoparticles,” which term is used interchangeably with the term “plasmon nanoparticles.” These plasmon supporting nanoparticles also can exhibit shifted and altered responses to electromagnetic waves when they are in the form of aggregates or have fractal structures. Examples of this effect may be observed in surface enhanced Raman Scattering. Gold and silver colloids have been shown to undergo strong color changes when they are concentrated due to interactions between colloid particles. These changes are illustrated, for example, in Michael Quinten: “Optical Effects Associated with Aggregates of Clusters”, Journal of Cluster Science, Vol. 10. No. 2, 1999. For example, for silver particles, the isolated particle sample appears yellow due to the surface plasmon, which is peaked at the wavelengths of blue light. The color of the aggregated samples changes, however, into orange, brown, and green as the amount of silver particles in the aggregate increases. For gold, the red color of the isolated particle sample changes for the aggregated sample into violet and blue as the amount of gold particles in the aggregate increases. The role of interparticle separation on the color has been demonstrated by Kotov et al. (J. Phys. Chem. (1995) 99, 13065) where multilayers of SiO₂ coated gold nanoparticles are formed. Particles with thicker shells are redish whereas particles which have thinner shells and are closer, are blue.

In accordance with certain examples, the exact nature and chemical makeup of the plasmon nanoparticles used in the exemplary pixels, displays and inks disclosed herein may vary depending on the desired color, or colors, to be transmitted or reflected. In some examples, the plasmon nanoparticles are charged or receptive to being charged (e.g., positive, negative, a partial positive charge, a partial negative charge or a dipole), whereas in other examples, the plasmon nanoparticles are uncharged or neutral. In certain examples, a plasmon nanoparticle comprises a non-conductive material, a conductive material or a semi-conductive material. In some examples, the plasmon nanoparticle comprises two or more of a non-conductive material, a conductive material and a semi-conductive material. In examples where the plasmon nanoparticle includes a non-conductive material, the non-conductive material may be selected from one or more of titania, zinc oxide, clays, magnesium silicate, glasses or other suitable non-conductive materials. In examples where the plasmon nanoparticle includes a conductive material, the conductive material may be selected from metals, or combinations of metals, such as, for example, transition metals and alloys of these metals. In certain examples, the conductive material includes one or more of silver, gold, platinum, palladium, ruthenium, rhodium, osmium, iridium and alloys of these metals. In examples where the plasmon nanoparticle includes semi-conductive materials, the semi-conductive material may be selected from one or more of cadmium selenide, cadmium telluride, zinc selenide, zinc telluride, cadmium phosphide, cadmium arsenide, gallium selenide, aluminum arsenide and the like. It will be recognized by the person of ordinary skill in the art, given the benefit of this disclosure, that the optical characteristics of a pixel, display or ink may vary depending on the composition of the plasmon nanoparticles and that non-conductive nanoparticles, conductive nanoparticles and semi-conductive nanoparticles may not provide the same optical response when aggregated or concentrated.

In accordance with certain examples, the exact size, e.g., diameter, of the plasmon nanoparticles used in the exemplary pixels, displays and inks disclosed herein may vary, but the particle size is typically much smaller than the wavelength of transmitted or reflected light. In certain examples, the smallest dimension of the diameter of a plasmon nanoparticle filled microcapsule is less than about 500 nm, more particularly less than about 200 nm or 100 nm, e.g., about 50 nm in diameter, 25 nm in diameter or less. Similarly, the exact form or topology of aggregates formed from the plasmon nanoparticles may vary and illustrative aggregate forms include, but are not limited to, fractal structures, linear forms, cross-shaped forms, T-shaped forms, trapezoid shaped forms, U-shaped forms, gamma shaped forms, corner shaped forms or other suitable forms that the aggregate may adopt. The concentration of the plasmon nanoparticles may vary depending on the intended use, e.g., pixel, ink, etc., and the particular chemical makeup of the plasmon nanoparticles. Suitable plasmon nanoparticle concentrations include but are not limited to those concentrations that are effective to bring the particles to within an average distance of a few diameters, e.g., before, during or after a perturbation, to very dilute concentrations where the particles are separated by over about a wavelength. Additional suitable sizes, forms and concentrations will be readily selected by the person of ordinary skill in the art, given the benefit of this disclosure.

In accordance with certain examples, using plasmon nanoparticles (including core-shell structures), a perturbation may be applied to the plasmon nanoparticles to concentrate or focus the plasmon nanoparticles in a particular region, e.g., to increase the local concentration of nanoparticles in a particular region, such that an aggregate of the plasmon nanoparticles forms. Without wishing to be bound by any particular scientific theory, the perturbation is operative to cause color transmission or absorption changes in the plasmon nanoparticles as the plasmon nanoparticles aggregate, e.g., as the concentration of plasmon nanoparticles in the aggregate increase. In certain examples, the perturbation allows the transmitted or reflected color to continuously vary over a wavelength range, e.g., from red to violet. As used herein, when the color “continuously varies,” the color may be any color within a particular wavelength range including the end-point colors. In an illustrative example, when the transmitted or reflected color varies continuously in the visible wavelength range, the transmitted or reflected color may be any wavelength between about 380 nm and about 800 nm, for example. As the wavelength of light varies, e.g., changes from red to blue, it need not pass through the wavelengths of light in between. That is, the transmitted or reflected light may simply change from a first wavelength to a second wavelength without passing through wavelengths in between the first wavelength and the second wavelength. In certain examples, when the perturbation is removed, the concentration of plasmon nanoparticles in the aggregate decreases as the plasmon nanoparticles dissociate or return to their pre-perturbation state or some other non-aggregated form.

In accordance with certain examples, the exact nature of the perturbation may vary depending on the device or material that uses the plasmon nanoparticles. In certain examples, the perturbation is an external field, such as an electric field or a magnetic field. In other examples, the perturbation is an acoustic wave. In yet other examples, the perturbation is caused by a field gradient, e.g., an electric, magnetic, or acoustic gradient. In still other examples, the perturbation may be a temperature, pressure or concentration gradient. In additional examples, the perturbation may be other physiochemical stimuli that are operative to focus or concentrate nanoparticles. In the case of neutral nanoparticles, neutral nanoparticles may be focused or concentrated using, for example, field gradients or thermophoretic forces. Additional methods of perturbing charged and/or uncharged nanoparticles will be readily selected by the person of ordinary skill in the art, given the benefit of this disclosure. The perturbation may be applied using numerous methodologies including continuous application, intermittent application, pulsed application and the like. The intensity or strength of the perturbation may vary or may be constant.

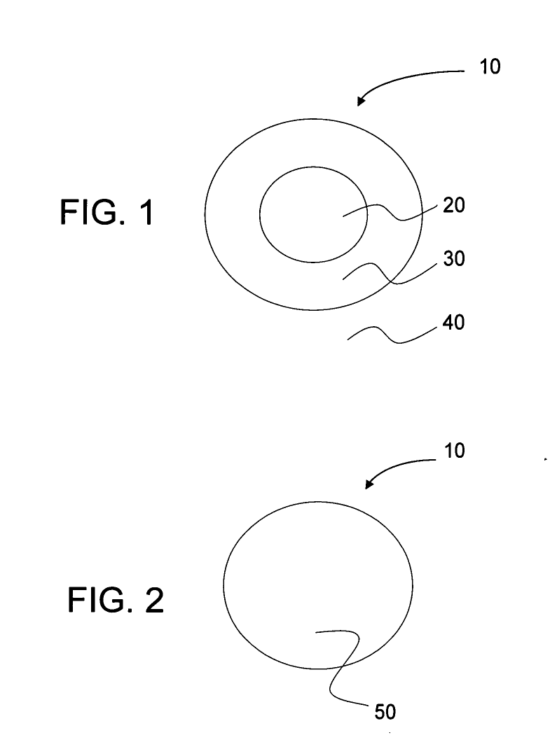

In accordance with certain examples and referring to FIG. 1, an example of a core-shell plasmon supporting nanostructure 10 is disclosed. While the nanoparticle in FIG. 1 is shown as substantially spherical, core-shell plasmon nanoparticles useful in the illustrative pixels, displays and inks disclosed herein may be non-spherical and may be symmetric or asymmetric. In certain examples, the plasmon nanoparticles are elliptical, spheroid, triangular, rectangular, or may take other suitable geometries commonly found in atomic and molecular structures. In some examples, the plasmon nanoparticle may include an electrically conductive shell around an insulating core, or an electrically insulating shell around a conductive core. For example, an insulating core may be formed from non-conductive materials such as those described herein. In certain examples and referring to FIG. 1, a plasmon nanoparticle 10 comprises an inner medium 20, which may be, for example, a metal or a dielectric. The plasmon nanoparticle 10 also comprises an outer medium 30, which may be, for example, a dielectric or metal that surrounds the inner medium 10. The plasmon nanoparticle may also include an external medium 40, which is a surrounding dielectric medium. In certain examples, the dielectric for any one or more of media 20, 30 or 40 may be a fluid, such as a gas, liquid, supercritical fluid and the like. In some examples, the dielectric is selected from one or more of materials that are non-conductive at the frequencies (or wavelengths) of interest or is a material which does not posses a negative real dielectric constant. Illustrative examples of dielectric materials suitable for use in pixels, displays and inks include, but are not limited to, oxides, such as TiO₂, ZnO, SiO₂, or polymeric materials such as PMMA or styrene. Depending on the material properties, size and shape geometries of these core-shell plasmon nanoparticles, they can be made to exhibit a specific plasmon resonance. Plasmon nanoparticles may also be made of a single medium of material 50, e.g., a metal, as shown in FIG. 2. The person of ordinary skill in the art, given the benefit of this disclosure, will be able to select and/or design suitable plasmon nanoparticles for use in the illustrative pixels, displays and inks disclosed herein. Exemplary nanoparticles suitable for use in the pixels, displays and inks disclosed herein include, but are not limited to, those described in Liz-Marzan, L. M. “Nanometals: Formation and Color.” Materials Today, pp. 26-31 (February 2004). Illustrative methods for producing nanoparticles include, but are not limited to, those methods described in U.S. Pat. No. 5,882,779, the entire disclosure of which is hereby incorporated herein by reference for all purposes.

In accordance with certain examples, plasmon nanoparticles suitable for use in the pixels, displays and inks disclosed herein may also include modified surfaces. For example, the surface of a plasmon nanoparticle may be modified to be magnetic, modified to have charged and/or uncharged groups, modified to render the nanoparticle asymmetric or anisotropic, or may be modified in other suitable manners using suitable chemical reagents, such as those commonly used to accomplish chemical surface modification. Without wishing to be bound by any particular scientific theory, the use of anisotropic plasmon nanoparticles may also lead to polarization sensitive concentration color effects which may be useful for pixels, displays and inks.

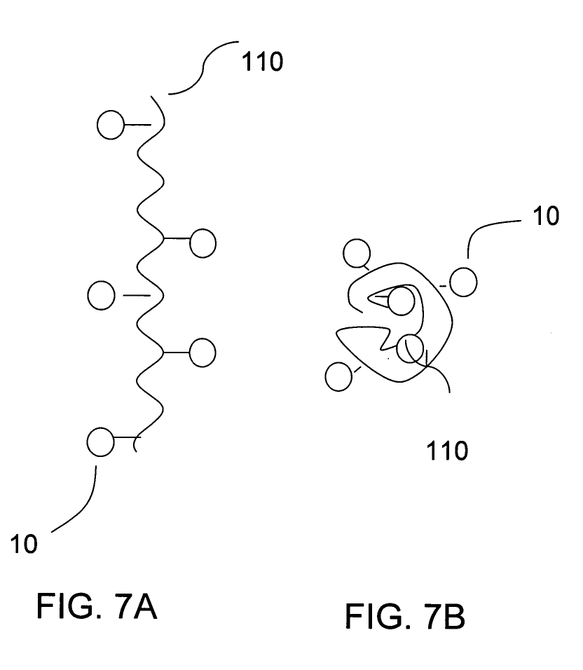

In accordance with certain examples, an aggregating or concentrating agent may be present with the plasmon nanoparticles. As used herein an “aggregating agent” or “concentrating agent” promotes or drives the plasmon nanoparticles to be closer in space in response to a perturbation. The exact nature and concentration of the concentrating agent may vary depending on whether the plasmon nanoparticles are used in pixels, inks or other devices or compositions and depending on the exact chemical makeup of the plasmon nanoparticles. In certain examples, the concentrating agent may be a chemical agent such as a lower critical solution temperature (LCST) material or photoacid. In some examples, the concentrating agent is a biological agent such as a polynucleotide, a polypeptide, a polysaccharide, a lipid, a phospholipid, a fatty acid or the like to which the particle is attached. For example, the use of DNA and other molecules that can change or alter their conformation, e.g., proteins, to drive aggregation with an external stimulus is possible such as an electric field. One example may be found in Michael J. Heller, “Electric Field Assisted Self-Assembly of DNA Structures: A Potential Nanofabrication Technology” given at the Sixth Foresight Conference on Molecular Nanotechnology in 1998. In particular, as the biomolecule transitions, for example, from a linear to a coiled state as a function of an applied field or other physiochemical stimuli (e.g., pH, ionic strength changes), aggregate formation of plasmon nanoparticles may be promoted. For example, referring to FIG. 7A, a concentrating agent 110, e.g., a biomolecule, comprises plasmon nanoparticles 10, which are associated with concentrating agent 110. The plasmon nanoparticles 10 may be covalently bound to the concentrating agent, may interact with the concentrating agent through one or more salt bridges or ionic bonds, may interact through partial positive charges, partial negative charges or other suitable chemical or physical interactions to reversibly or irreversibly associate the plasmon nanoparticles with the concentrating agent. Referring now to FIG. 7B, as the concentrating agent changes from a first, uncoiled state (as shown in FIG. 7A) to a second, coiled state, the plasmon nanoparticles are brought closer together such that the local concentration of the plasmon nanoparticles increases. It will be within the ability of the person of ordinary skill in the art, given the benefit of this disclosure to select suitable concentrating agents for an intended use.

In accordance with certain examples, a pixel comprising a plurality of plasmon nanoparticles is provided. In certain examples, the pixel is configured to transmit or to reflect a variable wavelength of light with varying concentrations of the plasmon nanoparticles. For example, as the concentration of plasmon nanoparticles in an aggregate increases, the wavelength of light transmitted or reflected by a pixel changes. Depending on the exact configuration of a device that includes a pixel, the pixel may transmit the variable wavelength of light or may reflect the variable wavelength of light, e.g., the pixel may transmit or reflect any wavelength of light within a selected or desired wavelength range. In certain examples, the color transmitted by the pixel may continuously vary over a wavelength range from the infrared light to red, orange, yellow, green, blue and violet light or even ultraviolet light depending on the nature and concentration of plasmon nanoparticles. As discussed herein, the color of the pixel is tunable by varying the concentration of plasmon nanoparticles using, for example, a perturbation to increase or decrease the local concentration of plasmon nanoparticles in a particular region.

In some examples, the plasmon nanoparticles in the pixel are conductive materials, non-conductive materials or semi-conductive materials as disclosed herein. In other examples, the plasmon nanoparticles include core-shell materials as described herein. Regardless of the form and nature of the plasmon nanoparticles, the plasmon nanoparticles may remain free within the pixel or may be encapsulated to form plasmon nanoparticles filled microcapsules within the pixel. As used herein, “plasmon nanoparticles filled microcapsules” refer to structures having some boundary or barrier to contain plasmon nanoparticles within, e.g., capsules, micelles, liposomes, membranes or the like. When plasmon nanoparticles are encapsulated to form microcapsules, the smallest dimension of microcapsule is typically less than the wavelength of the transmitted light or the reflected light as described before. The capsules themselves, however, have no size restrictions and may comprise a single pixel.

In some examples, the plasmon nanoparticle filled microcapsules are individually tunable over a visible wavelength range, e.g., the color transmitted or reflected by each microcapsule may be any color within an infrared, visible or ultraviolet wavelength range. This individual tuning of microcapsules, e.g., individual tuning of the absorption, scattering or transmission response of the microcapsule, allows for numerous shades of colors and numerous color combinations. In certain examples, the plurality of microcapsules each may comprise different plasmon nanoparticles such that as the microcapsules concentrate, e.g., after application of a suitable perturbation, the transmitted color is a combination of the colors transmitted or reflected by the individual microcapsules. By assembling a pixel that include plasmon nanoparticles, and/or microcapsules, that have different optical responses after application of a perturbation, the entire spectrum of colors in the visible wavelength range may be transmitted or reflected by the pixel. This feature may provide for significant enhancement in color contrast, resolution, and the like. It will be within the ability of the person of ordinary skill in the art, given the benefit of this disclosure, to design suitable pixels that include plasmon nanoparticles and microcapsules.

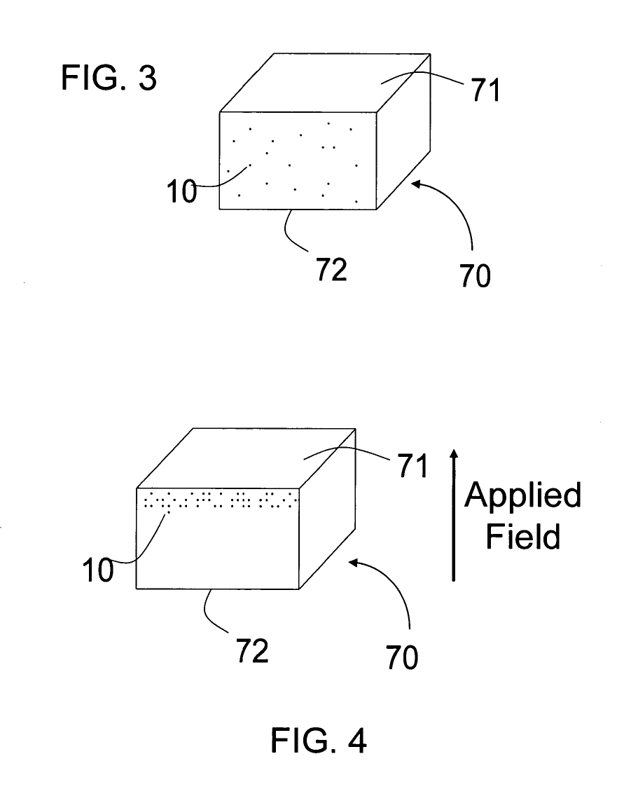

In accordance with certain examples, the pixels disclosed herein may be configured similarly to pixel configurations known in the art. For example and referring to FIG. 3, a pixel 70 may include a transmissive surface 71, a second reflective or transmissive surface 72, and a plurality of plasmon nanoparticles 10 disposed between the surfaces 71 and 72. The schematic shown in FIG. 3 represents a pixel in a first state where no perturbation has been applied. The plasmon nanoparticles 10 are randomly dispersed between the surfaces 71 and 72 when no perturbation is applied. In the first state, the color, if any, provided by the pixel 70 may represent the color transmitted or reflected from the disaggregated, random state of the plasmon nanoparticles 10.

Referring now to FIG. 4, the pixel 70 of FIG. 3 is now shown after application of a perturbation, which is an external electric field in this example. The perturbation causes the plasmon nanoparticles 10 to aggregate or concentrate near or adjacent to the transmissive surface 71 in a second state. As discussed herein, aggregation of the plasmon nanoparticles provides a change in the wavelength of light transmitted or reflected by the pixel 70, e.g., the transmitted or reflected light may change from a first wavelength to a second wavelength. While not shown in the figures, removal of the perturbation allows for return of the plasmon nanoparticles 10 to the first state as shown in FIG. 3. In some examples, surfaces 71 and 72 may each be configured as electrodes such that a perturbation can be applied using the surfaces 71 and 72. It will be within the ability of the person of ordinary skill in the art, given the benefit of this disclosure, to configure the surfaces of a pixel as electrodes.

In certain examples, an opposite configuration to the configuration just described may also be implemented. For example, a first state of a pixel may exist where the plasmon nanoparticles aggregate or concentrate near or adjacent to the transmissive surface 71 due to an intrinsic charge on the transmissive surface 71. A perturbation may be applied to convert the pixel from the first state to a second state where the aggregated plasmon nanoparticles disperse or disaggregate, which would alter the wavelength of light transmitted or reflected by the pixel. To minimize the amount of external power required, it may be desirable to implement this configuration when the wavelength of light provided by the pixel in the first state is to be maintained for significant periods, e.g., in multi-color lighted displays or lighted signs, and the wavelength of light provided in the second state is infrequent. Alternatively, within a range of physiochemical parameters, the aggregation may be stable and hence not require the continued application of a perturbation to maintain the aggregate state. In this case, a second application of a perturbation, which may be the same or different as the first application, may be used to reverse concentration or aggregation of the plasmon nanoparticles resulting in a return to the more separated particle optical properties. It will be within the ability of the person of ordinary skill in the art, given the benefit of this disclosure, to design suitable pixels including plasmon nanoparticles.

In accordance with certain examples, the pixel may also include additional components and devices necessary to apply a perturbation or necessary to provide a desired wavelength of light. For example, the pixel may include electrodes for applying electric fields and/or magnetic fields or for creating temperature gradients, may include sound wave or pressure generators or may include additional devices configured to apply suitable perturbations to the plasmon nanoparticles, or microcapsules, in the pixel. One or more surfaces of the pixel may also include a filter or material configured to remove unwanted ultraviolet light reflections, or ultraviolet light transmissions, from the light reflected or transmitted by the pixel. One or more surfaces may include polarizers or materials configured to polarize the light. Additional components and devices useful with the pixels disclosed herein will be readily selected by the person of ordinary skill in the art, given the benefit of this disclosure.

In accordance with certain examples, the pixels disclosed herein may be illuminated from a top surface of the pixel or may be illuminated from a bottom or back surface of the pixel. For example, if illuminated from the top surface, the pixel may be considered passive and may locally change colors in a pixelated format. Similarly, if illuminated from the back surface, the effect may be used to affect the transmission of various light sources to create the image. A significant advantage of the pixels provided herein is the ability to tune the color of the pixel continuously through varying levels of plasmon nanoparticle concentrations, e.g., where the pixel is tunable in the visible wavelength range, the pixel may transmit or reflect any color between, and including, red and violet. Suitable light sources for illuminating the pixels will be readily selected by the person of ordinary skill in the art, given the benefit of this disclosure, and exemplary light sources include, but are not limited to, lamps, e.g., lamps emitting visible light, and light sources commonly used in liquid crystal displays.

In accordance with certain examples, a display comprising a plurality of pixels is provided. In certain examples, at least one of the pixels in the display comprises a pixel configured to transmit or to reflect a variable wavelength of light with varying concentrations of plasmon nanoparticles. In some examples, each of the plurality of pixels of the display comprises a plurality of plasmon nanoparticles, as described herein. Each of the pixels of the display may be constructed as described herein, or may be constructed using additional suitable methods that will be readily selected by the person of ordinary skill in the art, given the benefit of this disclosure. In certain examples, the display is configured as a flat panel display, e.g., a liquid crystal display. As discussed herein, each of the pixels may be configured to provide light that varies over a visible wavelength range, e.g., any wavelength between, and including, 380-800 nm.

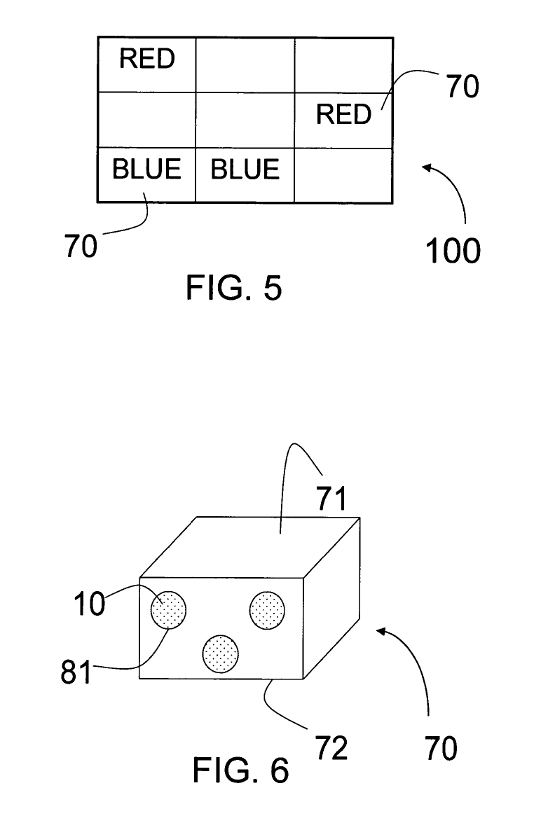

Referring to FIG. 5, a display 100 with a plurality of pixels 70 each with individually controllable ranges of plasmon nanoparticle concentrations is shown. In the example shown in FIG. 5, two of the pixels transmit or reflect red light and two of the pixels transmit or reflect blue light. As described herein, gold plasmon nanoparticle aggregates may provide a red color in a disaggregated state and the color transition towards blue with increasing amounts of larger aggregates in the pixel, e.g., by applying a perturbation to the pixel comprising the gold plasmon nanoparticles. The exact composition of the plasmon nanoparticles in each pixel of the display may vary and in certain examples may include non-conductive materials, conductive materials and/or semi-conductive materials. In certain examples, the plurality of plasmon nanoparticles in each pixel of the display may comprise one or more members selected from the group consisting of silver, gold, platinum, palladium, ruthenium, rhodium, osmium, iridium, and alloys thereof.

In accordance with certain examples, the plasmon nanoparticles in each pixel of the display may be encapsulated to form a plurality of microcapsules. For example, FIG. 6 shows an example of a pixel 80, suitable for use in a display, with microcapsule 81, which contains a plurality of plasmon nanoparticles 10. In certain examples, each of the plurality of microcapsules within each pixel is tunable over an infrared wavelength range, a visible wavelength range or an ultraviolet wavelength range. In some examples, each of the plurality of microcapsules within each pixel may comprise different plasmon nanoparticles, and, in certain examples, at least one of the plurality of microcapsules includes silver or gold.

In accordance with certain examples, the display may also include suitable additional components and devices. For example, the display may include a lamp or light source for illuminating the pixels. The display may also include suitable polarizers, such as those found in liquid crystal displays. The display may include a power supply and suitable interfaces for receiving signals, e.g., signals from a graphics card, a television tuner or the like. It will be within the ability of the person of ordinary skill in the art, given the benefit of this disclosure, to design suitable displays using the pixels disclosed herein.

In accordance with certain examples, an ink comprising a plurality of plasmon nanoparticles is disclosed. In certain examples, the color of the ink is continuously variable over a wavelength range with varying plasmon nanoparticles or with varying concentrations of the plasmon nanoparticles. For example, the ink color may vary from red, orange, yellow, green, blue, violet or any color in between. In other examples, the color of the ink is variable over an infrared wavelength range. In yet other examples, the color of the ink is variable over an ultraviolet wavelength range (e.g., about 10 nm to about 380 nm) with varying concentrations of the plasmon nanoparticles. In certain examples, the ink absorbs UVA (320-380 nm) or UVB (280-320 nm) light when illuminated with a suitable light source, such as a black light. The inks disclosed herein that use aggregated or concentrated plasmon nanoparticles can provide any color over an entire wavelength range of infrared, visible and ultraviolet light, do not require encapsulation and do not require any electrophoretic forces. In certain examples, the plurality of plasmon nanoparticles may be encapsulated to form a plurality of plasmon nanoparticle filled microcapsules. Each of the plurality of microcapsules may be tunable over a wavelength range. In addition, the plurality of microcapsules may include different plasmon nanoparticles, e.g., silver and gold.

In certain examples, the plasmon nanoparticles, or the microcapsules as the case may be, can be placed in a carrier prior to use as an ink. For example, the microcapsules shown in FIG. 6, which contain a plurality of plasmon nanoparticles 10 contained within the microcapsule 81, may be placed into a suitable ink carrier for printing. Suitable carriers will be readily selected by the person of ordinary skill in the art, given the benefit of this disclosure and illustrative carriers include, but are not limited to, paste ink vehicles (which may consist of a small amount of solvent and/or phenolic resins, and/or alkyd resins, and/or nitrocellulose, and/or rosin maleic ester, and/or thinning oils, and/or waxes, and/or metal salt driers), UV curing type ink carriers, UV curing type inks carrier that are variable in viscosity and are free radical vehicles which may consist of about 5-80% acrylated oligomer(s) such a acrylated polyurethanes, acrylated polyesters, and acrylated epoxies, 5-90% acrylated monomer(s) such as 1,6-hexanedioldiacrylate, or alkoxylated tetrahydrofurfuryl acrylate, or trimethylolpropane trimethylacrylate, 0.1-10% photoinitiator(s) such as derivatives of benzophenone, phosphine oxides, 0-10% amine synergist and 0-20% adhesion promoters such as multifunctional acid esters (all of which are commercially available from Sartomer Company, Inc. (Exton, Pa.)). Examples of resin blends and dispersion vehicles that are suitable for use as carriers include, for example, those commercially available from Lawter Intl., Inc. (Pleasant Prairie, Wis.). Additional carriers suitable for use with the plasmon nanoparticles to provide inks will be readily selected by the person of ordinary skill in the art, given the benefit of this disclosure. The microcapsules in the carrier may then be disposed in a filled region and their optical response controlled through the aggregation, proximity or by the concentration of the microcapsules to impart a desired color response.

In accordance with certain examples, a method of tuning a pixel is disclosed. In certain examples, the method includes perturbing plasmon nanoparticle concentration to control color absorption or color transmission of the pixel along a wavelength range. In certain examples, the pixel is perturbed to concentrate the plasmon nanoparticles to provide a continuously variable color absorption or color transmission along a wavelength range, e.g., a distinct color absorption or color transmission response for the pixel. As discussed herein, in certain examples the perturbing step may be performed by applying numerous forces including, but not limited to, electric fields, magnetic fields, acoustic waves, field gradients, thermophoretic forces and the like. In certain examples, the color transmitted, or reflected, by the pixel may be configured to be one or more of red light, orange light, yellow light, green light, blue light, violet light or any wavelength of light between these colors. In other examples, the transmitted or reflected color may be in the infrared range or in the ultraviolet range. In certain examples, the plasmon nanoparticles may be configured to form a plurality of plasmon nanoparticle filled microcapsules. In additional examples, each of the plurality of microcapsules may be configured to be tunable over a visible, infrared, or ultraviolet wavelength range (or combinations thereof). In yet other examples, each of the plurality of microcapsules may be configured to comprise different plasmon nanoparticles. In still other examples, one or more of the plurality of microcapsules may be configured to include silver or gold. In some examples, the pixel, or ink as the case may be, may be configured with an concentrating or aggregating agent to promote aggregation of the plasmon nanoparticles. Examples of suitable concentrating agents are disclosed herein.

In accordance with certain examples, a method of facilitating control of a pixel in a display by providing a pixel configured to transmit or to reflect a variable wavelength of light with varying concentrations of plasmon nanoparticles. The plasmon nanoparticles may be free or may be encapsulated to form microcapsules. It will be within the ability of the person of ordinary skill in the art, given the benefit of this disclosure, to facilitate control of the pixels, displays and inks disclosed herein.

When introducing elements of the examples disclosed herein, the articles “a,” “an,” “the” and “said” are intended to mean that there are one or more of the elements. The terms “comprising,” “including” and “having” are intended to be open ended and mean that there may be additional elements other than the listed elements. It will be recognized by the person of ordinary skill in the art, given the benefit of this disclosure, that various components of the examples can be interchanged or substituted with various components in other examples. Should the meaning of the terms of any of the patents, patent applications or publications referred to herein conflict with the meaning of the terms used in this disclosure, the meaning of the terms in this disclosure are intended to be controlling.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

1. A pixel comprising a plurality of plasmon nanoparticles in the pixel.

2. The pixel of claim 1 in which the pixel is configured to transmit or to reflect a variable wavelength of light with varying concentrations of the plasmon nanoparticles.

3. The pixel of claim 2 in which the wavelength of light is continuously variable over an infrared wavelength range, a visible wavelength range or an ultraviolet wavelength range.

4. The pixel of claim 2 in which the plasmon nanoparticles comprise one or more members selected from the group consisting of silver, gold, platinum, palladium, ruthenium, rhodium, osmium, iridium, and alloys thereof.

5. The pixel of claim 1 in which the plasmon nanoparticles comprise one or more members selected from the group consisting of titania, zinc oxide, and glasses.

6. The pixel of claim 1 in which the plasmon nanoparticles comprise one or more members selected from the group consisting of cadmium selenide, cadmium telluride, zinc selenide, zinc telluride, cadmium phosphide, cadmium arsenide, gallium selenide, and aluminum arsenide.

7. The pixel of claim 1 in which the plasmon nanoparticles comprise at least one core-shell plasmon supporting nanostructure.

8. The pixel of claim 1 in which the plasmon nanoparticles comprise gold or silver.

9. The pixel of claim 8 in which the plurality of plasmon nanoparticles are encapsulated to form at least one plasmon nanoparticle filled microcapsule.

10. The pixel of claim 9 in which the a plasmon nanoparticle filled microcapsule provides an absorption, scattering or transmission response which is tunable over a wavelength range.

11. The pixel of claim 1 in which the plurality of plasmon nanoparticles are encapsulated to form a plurality of a plasmon nanoparticle filled microcapsules.

12. The pixel of claim 11 in which each of the plurality of plasmon nanoparticle filled microcapsules is tunable over an infrared wavelength range, a visible wavelength range, or an ultraviolet wavelength range.

13. The pixel of claim 11 in which the plurality of plasmon nanoparticle filled microcapsules comprise different plasmon nanoparticles.

14. The pixel of claim 13 in which one or more of the microcapsules comprises silver or gold.

15. The pixel of claim 1 further comprising a concentrating agent.

16. The pixel of claim 10 further comprising a concentrating agent in the plasmon nanoparticle filled microcapsule.

17. A display comprising at least one pixel of claim 1.

18. The display of claim 17, further comprising a plurality of pixels, wherein each of the plurality of pixels comprises the pixel of claim 1.

19. An ink comprising a plurality of plasmon nanoparticles, wherein the color of the ink is continuously variable over a wavelength range with varying plasmon nanoparticles or with varying plasmon nanoparticle concentrations.

20. The ink of claim 19 in which the plurality of plasmon nanoparticles are encapsulated to form a plurality of plasmon nanoparticle filled microcapsules.

21. The ink of claim 19 in which the plurality of plasmon nanoparticle filled microcapsules comprise different plasmon nanoparticles.

22. The ink of claim 19 in which the wavelength range is an ultraviolet wavelength range, a visible wavelength range or an infrared wavelength range.

23. The ink of claim 19 further comprising a concentrating agent.

24. The ink of claim 20 further comprising a concentrating agent in at least one of the plurality of plasmon nanoparticle filled microcapsules.

25. The ink of claim 19 further comprising a carrier.

26. A method comprising perturbing a plasmon nanoparticle concentration to control color absorption or color transmission of a pixel along a wavelength range.

27. The method of claim 26 further comprising configuring the color absorption or the color transmission to be continuously variable along the wavelength range.

28. The method of claim 26 further comprising applying one or more of an electric field, a magnetic field, an acoustic wave, a field gradient or thermophoretic forces to perturb the plasmon nanoparticle concentration.

29. A method of facilitating control of a pixel in a display by providing a pixel configured to transmit or to reflect a variable wavelength of light with varying concentrations of plasmon nanoparticles.

30. The method of claim 29 in further comprising configuring the plasmon nanoparticles to be in plasmon nanoparticle filled microcapsules.
